(19) United States
(12) Patent Application Publication
(10) Pub. No.: US 2013/0090237 A1
(43) Pub. Date: Apr. 11, 2013

(54) PTEREDIN PENTANEDIOIC DERIVATIVE BASED NANOPARTICLES

(71) Applicant: Sanat Mohanty, New Delhi (IN)

(72) Inventor: Sanat Mohanty, New Delhi (IN)

(21) Appl. No.: 13/270,307

(22) Filed: Oct. 11, 2011

(51) Int. Cl. A01N 25/02; A61K 9/08; A61K 47/24; A61K 47/34; A61K 47/32; A61K 47/26; A61K 47/38; A61K 47/36; A01P 1/00; A01P 13/00; A01N 61/00; A01N 33/00; B05D 7/24; B05D 3/02; A61K 47/22; B82Y 5/00; B82Y 40/00

(52) U.S. Cl. 504/118; 514/788; 424/400; 424/405; 514/772.6; 514/772.5; 514/772.4; 514/772.3; 514/777; 514/780; 514/781; 514/778; 504/358; 427/226; 977/915; 977/890

(57) ABSTRACT

In one embodiment, a method includes making a pteredin phenyl pentanedioic (3P) formulation by providing an aqueous solution including one of more 3P molecules neutralized with one or more of an alkali, an alkali earth metal hydroxide, or an alkali carbonate; adding to the aqueous solution one of a surfactant, dispersant, or additive with the guest molecules; and non-covalently crosslinking the 3P formulation by exposing the 3P formulation to an excess solution of multivalent cation salt.

### TECHNICAL FIELD

This disclosure generally relates to nanoparticles.

### BACKGROUND

Nanoparticles having a diameter between 1 to 1000 nanometers (nm), may be used for a variety of applications including in diagnostics, drug delivery, and optical and smart materials. Nanoparticles have been developed from metals, inorganic oxides, carbon nano-structures, polymer chains and dendrites as well as liquid crystalline and vesicular structures. Effective bioactive delivery encapsulates and protects a bioactive molecule in incompatible environments. It requires the carrier be non-toxic and compatible with body fluids of interest, support potential targeting, and allow for trigger or stimulus based release. The choice of drug delivery agent and strategy of encapsulation and delivery can thus affect effectiveness of the drug.

### BRIEF DESCRIPTION OF DRAWINGS

FIG. 1 illustrates structures of example pteredin-pentanedioic derivatives.

FIG. 2 illustrates foliate particles in foliate liquid crystalline solution.

### DETAILED DESCRIPTION OF THE EXAMPLE EMBODIMENTS

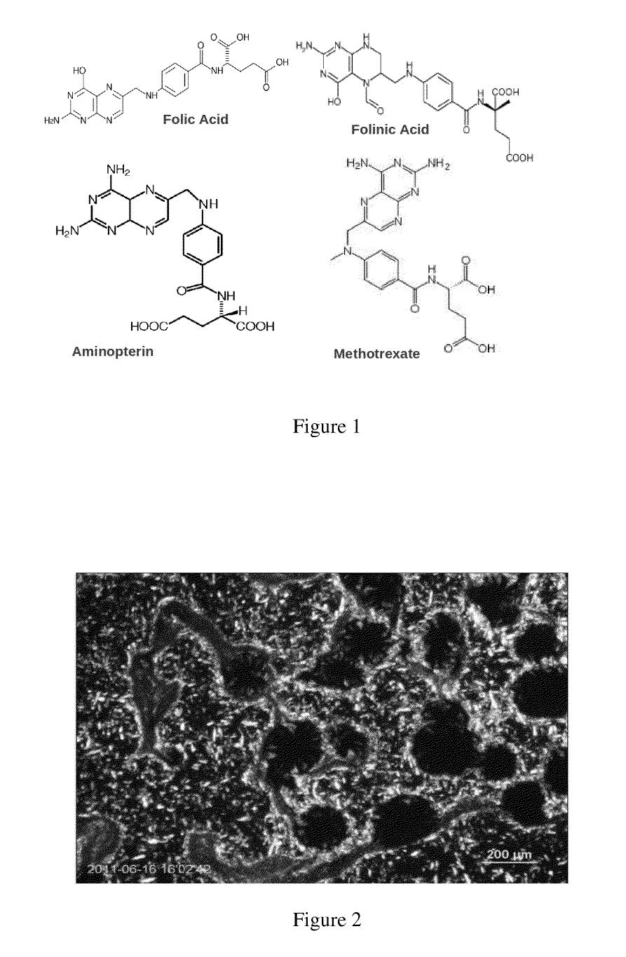

FIG. 1 illustrates molecular structures of example pteredin-pentanedioic (3P) derivatives. As an example and not by way of limitation, pteredin-phenyl-pentanedioic derivatives may include folic acid ((2S)-2-[(4-{[(2-amino-4-hydroxypteridin-6-yl)methyl]amino}phenyl)formamido]pentanedioic acid), folinic acid ((2S)-2-{[4-[(2-amino-5-formyl-4-oxo-5,6,7,8-tetrahydro-1H-pteridin-6-yl)methylamino]benzoyl]amino}pentanedioic acid), aminopterin ((2S)-2-[[4-{[(2,4-Diaminopteridin-6-yl)methyl]amino}benzoyl]amino]pentanedioic acid) and methotrexate ((2S)-2-[(4-{[(2,4-diaminopteridin-6-yl)methyl](methyl)amino}benzoyl)amino]pentanedioic acid). Many 3P derivatives may be used as therapeutics and dietary supplements and may be candidates for high yield encapsulating agents for guest molecules. As an example and not by way of limitation, folic acid may be contained in vegetables and in particular embodiments, folic-acid based supplements may be taken without any known toxicity concerns. In addition, folic acid-based functional groups may help target cancerous cells.

3P derivatives at concentrations approximately between 60% and 1% by weight may be dissolved in room temperature water. Dissolved 3P may be converted into a liquid crystalline phase with the addition of a pH-adjusting compound to modify the pH to a range between 6 and 8. As an example and not by way of limitation, pH-adjusting compounds may include sodium, potassium, lithium, ammonium hydroxide, various amines, any known base. The base may allow 3P derivatives to become more soluble in the aqueous solution and form a liquid crystalline phase. The shape and structure of nanoparticles may be affected by a concentration of the 3P derivatives in the final solution. Monovalent salts of 3P derivatives may self-assemble into cholesteric and hexagonally arranged stacked columns. In particular embodiments, monovalent salts of folic acid may self-assemble into cholesteric and hexagonally arranged stacked columns.

Guest molecules may be dissolved into 3P liquid crystalline solutions. The guest molecule may be added as a solute into 3P liquid crystalline solution or added after being made into an aqueous solution. In either case, an additive may be included to promote solubility of a drug into a 3P liquid crystalline matrix. Concentrations of the guest molecule may range from 50% to 0.1% by weight (or lower), and as an example but not by way of limitation, less than 25% by weight. As an example and not by way of limitation, guest molecules may include dyes, cosmetic agents, fragrances, flavoring agents, and bioactive compounds, such as drugs, herbicides, pesticides, pheromones, and antifungal agents. A bioactive compound is herein defined as a compound intended for use in the diagnosis, cure, mitigation, treatment or prevention of disease, or to affect the structure or function of a living organism. As an example and not by way of limitation, drugs intended to have a therapeutic effect on an organism (i.e. pharmaceutically active ingredients) may be particularly useful guest molecules. Alternatively, herbicides and pesticides are examples of bioactive compounds intended to have a negative effect on a living organism, such as a plant or pest. Other particular embodiments may include drugs that may be relatively unstable when formulated as solid dosage forms, drugs that may be adversely affected by the low pH conditions of the stomach, drugs that may be adversely affected by exposure to enzymes in the gastrointestinal tract, and drug that may be provided to a patient via sustained or controlled release.

In particular embodiments, an aqueous suspension of nanoparticles may be added to the aqueous solution of 3P derivatives described above. As an example and not by way of limitation, a nanoparticle may be made of metal (magnetic or non-magnetic), polymer, hydrogel, organogel, biomolecules, 3P based, imidazole based chromonic structures, lipids, or amphiphiles. In other particular embodiments, nanoparticles may contain a bioactive guest. The aqueous suspension of nanoparticles may further include additives (e.g. surface modifying agents, dispersants, viscosity modifiers or fillers specific to the nanoparticle of interest). In particular embodiments, nanoparticles added to the 3P liquid crystalline solution may contain guest molecules.

Solution of 3P derivatives along with additives, fillers and bioactive guests (herein referred to as the “3P formulation”) may be crosslinked, where non-covalent interactions may be used to bind 3P molecules to each other. These non-covalent interactions may be ionic (as a result of divalent, trivalent or higher multivalent cations) or through coordination complexes with metals. Cross-linking may be achieved by exposing the 3P liquid crystalline material containing guest molecules, nanoparticles, and additives to a cross-linking solution. Surfactants may promote solution of drug molecules into the 3P structure. Surfactants may be ionic and non-ionic (preferably, non-ionic). Optional additives such as viscosity modifiers (for example, polyethylene glycol) or binders (for example, low molecular weight hydrolyzed starches) may also be added.

A cross-linking solution is prepared by dissolving salt of one or more multivalent cations (such as Ca²⁺, Mg²⁺, Zn²⁺, Al³⁺, Fe³⁺) in water. The concentration of the solution may be from 1% to 30% but preferably from 5% to 20% by weight. Different cation types may be mixed to give an non-integer average cation valency. In particular embodiments, a mixture of divalent and trivalent cations may cause a slower dissolution rate than a solution where all of the cations are divalent. Coordinating cations may result in slower release of guest molecules. As an example and not be way of limitation magnesium, a non-coordinating divalent cation, may lead to faster release of guest molecules than calcium or zinc, both of which are coordinating divalent cations. The rate of release of guest molecules and the nature of sustained release may be controlled by the concentration and identity of cations in the crosslinking solutions as well as the time for cross-linking. Mixtures of 3P phases crosslinked with different cation sets may be used to provide a multimodal release protocol depending on the environment. In particular embodiments, cross-linking with radioactive salts may be used for in situ diagnostics or for targeted chemotherapy.

The 3P formulation may be added to the cross-linking solution described above as drops or in any other suitable shape. This 3P formulation may be added to excess cross-linking solution. Typically, 1 part of the 3P formulation is added to 10 parts of cross-linked solutions. The 3P formulation may stay in the cross-linking solution for some time (between 1 hour to 24 hours). The cross-linking solution maintains the macroscopic structure of the 3P phase in the absence of shearing, stirring or other external forces. The cross-linking solution may also allow the multivalent ions to cross-link 3P self-assemblies, thereby encapsulating the guests, nanoparticles or the additives within the 3P phase. 3P phases may be separated from the cross-linking solution by physical separation methods (e.g. filtration, decantation, centrifugation, etc) and dried or freeze dried thereby protecting the encapsulated bioactive guest molecule during storage.

Cross-linked 3P phases containing fillers or bioactive guest molecules may be placed in or exposed to solutions of monovalent cation salts (e.g. sodium chloride). The monovalent cations exchange with the multivalent cations in the cross-linked phase and the bioactive guest molecule may be released over time into the solution. Similar release may also be achieved by placing crosslinked 3P phase in a tris buffer solution or a phosphate buffer solution, which are also standard solutions with monovalent cations or amine groups.

The 3P formulation may be dispersed into a discontinuous phase by mixing the 3P phase with water soluble polymers with molecular weight less than 30000. In particular embodiments, 3P formulation may be mixed with water soluble polymers with molecular weight less than 20,000 (e.g., polyvinyl-based water-soluble polymers, polycarboxylates, polyacrylates, polyamides, polyamines, polyvinyl alcohol, polyethylene glycol, polypropylene glycol, poly(ethylene glycol)-co-(propylene glycol), polyglycols, cellulosics, starches (including modified starches such as phosphonated or sulfonated starches) and modified starches, and the like, and mixtures thereof). Copolymers, for example, block or random copolymers can also be useful.

The dispersed phase of 3P formulation may be between 10 nanometers (nm) to 5 mm. In the nano-scale, the dispersed phase may be between 10 nm to 1000 nm. The nanoscale dispersed phase may be spherical or acicular (i.e. with aspect ratio of 1:4 to 1:20). In other particular embodiments, nanoparticles of oblate spheroidal or toroidal shapes may be obtained. As an example and not by way of limitation dispersants may include alkyl phosphates, phosphonates, sulfonates, sulfates, or carboxylates. Carboxylates may include long chain saturated fatty acids or alcohols, mono or poly-unsaturated fatty acids or alcohols. In particular embodiments, oleyl phosphonic acid may be used as a dispersant.

The concentration of the polymer in the aqueous polymer solution as well as the ratio of polymer solution to the 3P formulation may be varied to control the size, size distribution or shape of the 3P formulation. The amount of 3P formulation may be controlled such that the 3P formulation may be in the discontinuous phase and the water soluble polymer may be the continuous phase in this two phase system. Amounts of water-soluble polymer and 3P may be selected for a ratio between 4:1 and 99:1 on a dry weight basis. In particular embodiments, the ratio of water-soluble polymer and folic acid may be in a range of 5:1 to 15:1 on a dry weight basis. In particular embodiments, the water-soluble polymer may comprise 10 to 25 weight % of the aqueous mixture and, the concentration of 3P may be from 0.25 to 20 weight % of the aqueous mixture.

Optionally, surfactants and other additives (for example, short chain alcohols such as ethanol) may be added to the aqueous mixture to increase surface tension or promote coating. Surfactants can also promote solution of drug molecules into the foliate structure. These surfactants may include ionic and non-ionic surfactants (preferably, non-ionic). Optional additives such as viscosity modifiers (for example, polyethylene glycol) or binders (for example, low molecular weight hydrolyzed starches) may also be added.

The polymer—3P formulation mixture may be placed in excess cross-linking solution and over time (e.g. 1 hour to 24 hours), the cross-linking solution may cause dispersion of the polymer phase and cross-linking of the 3P phase containing the bioactive guest molecules while keeping the shape of the 3P phase largely intact. After crosslinking, the 3P phase may be separated by physical separation processes from the crosslinking solution as described above. The 3P phase may then be dried or freeze dried.

A suspension of nanoparticles may be added to the aqueous solution of 3P. As an example and not by way of limitation, the nanoparticle may be made of metal, polymer, hydrogel, organogel, biomolecules, 3P based structures, imidazole based chromonic structures, lipids or amphiphiles and may contain a bioactive guest molecule. The suspension of nanoparticles may also include surface modifying agents specific to the nanoparticle of interest.

These nanoparticles may release their bioactive guest molecules with exposure to solutions of monovalent cation salts. In particular embodiments, guest molecules may have immediate or sustained release profiles. As an example and not by way of limitation, for immediate release use, most of the drug may be released in a short time (a range of time period of less than about 4 hours, to seconds). In other particular embodiments, for sustained (or controlled) release uses, most of the drug may be released in predefined and sustained rates over a longer period of time (e.g., from a few hours to a few weeks). A combination of immediate and sustained release profiles may release an initial burst to alleviate a particular condition followed by a sustained delivery to provide extended treatment of the condition.

In particular embodiments the nanoparticles may have a predefined drug release profile. An increasing or decreasing release profile may be used to match the daily rhythm of an organism. In other particular embodiments, solutions may have nanoparticles with different drug release characteristics. Alternatively, particles of the 3P formulation may be formed with a set of nanoparticles carrying one or more release profiles being contained in another nanoparticle.

The 3P formulation described above may selectively protect a drug from certain environmental conditions and controllably deliver the drug under certain environmental conditions. As an example and not by way of limitation, particles of the 3P formulation may be stable in the acidic environment of the stomach and dissolve when passed into the non-acidic environment of the intestine when administered to an animal, due to a change in pH. In particular embodiments, the particles of the 3P formulation may protect a drug from enzymatic degradation.

The 3P nanoparticles may isolate drug molecules in a particle, which may prevent unfavorable interactions (e.g., chemical reactions) between different drugs in a combination dosage form, unfavorable changes in a single drug component (e.g., Ostwald ripening or particle growth, changes in crystalline form), or unfavorable interactions between a drug and one or more excipients. As an example and not by way of limitation, a mixture of nanoparticles may allow two drugs that are ordinarily unstable in each other's presence to be formulated into a stable dosage form. In particular embodiments, a mixture of nanoparticles may allow a drug and excipient that are ordinarily unstable in each other's presence to be formulated into a stable dosage form.

The surfaces of the nanoparticles formed by 3P formulations may be modified to make the nanoparticle surface compatible to another formulation or attach preferentially to other surfaces. The cross-linked 3P nanoparticle may be isolated from the water-soluble polymer dispersion and re-suspended in a solution with a surface-modifying agent. Surface modifying groups may be derived from surface-modifying agents. Schematically, surface modifying agents may be represented by the formula A-B, where the A group may be capable of attaching to the surface of the 3P nanoparticle and the B group may be a compatibilizing group conferring hydrophilicity, hydrophobicity or biocompatibility to the surface modifying agent. Compatibilizing groups may be selected to control dispersability of the nanoparticles in a solvent of interest or adhesion to specific surfaces of interest. As an example and not by way of limitation, classes of surface-modifying agents include organic oxyacids of carbon, sulfur, phosphorus, and combinations thereof.

As an example and not by way of limitation polar surface-modifying agents having carboxylic acid functionality may comprise poly(ethylene glycol) monocarboxylic acid having the chemical structure CH₃O(CH₂CH₂O)ₙCH₂COOH (n=2-50) or 2-(2-methoxyethoxy)acetic acid having the chemical structure CH₃OCH₂CH₂OCH₂COOH in either acid or salt forms.

As an example and not by way of limitation, non-polar surface-modifying agents having carboxylic acid functionality may comprise octanoic acid, dodecanoic acid or oleic acid in either acid or salt form. In the case of a carboxylic acid containing olefinic unsaturation (e.g., oleic acid), the carbon-carbon double bonds may be present as either the Z or E stereoisomers or as a mixture thereof.

As an example and not by way of limitation phosphorus-containing acids may comprise alkylphosphonic acids, (e.g., octylphosphonic acid, decylphosphonic acid, dodecylphosphonic acid, octadecylphosphonic acid, oleylphosphonic acid or poly(ethylene glycol) monophosphonic acid having the chemical structure CH₃O(CH₂CH₂O)ₙCH₂CH₂PO₃H₂ (n=2-50) in either acid or salt forms). Phosphonic acids containing olefinic unsaturation, e.g. oleylphosphonic acid, may have carbon-carbon double bonds present as either the Z or E stereoisomers or a mixture thereof. As an additional example and not by way of limitation, phosphorus-containing acids may include alkyl phosphates, mono and diesters of phosphoric acid, octyl phosphate, dodecyl phosphate, oleyl phosphate, dioleyl phosphate, oleyl methyl phosphate, and poly(ethylene glycol) monophosphoric acid having a chemical structure CH₃O(CH₂CH₂O)ₙCH₂CH₂OPO₃H₂ (n=2-50).

In particular embodiments, the B group of the surface-modifying agent A-B may contain a functional group(s) to adjust either the hydrophilicity, hydrophobicity, or biocompatibility of the chromonic nanoparticle. As an example and not by way of limitation, functional groups may comprise hydroxyl, carbonyl, ester, amide, ether, amino, or quaternary-ammonium functions. Chromonic nanoparticles may be surface modified with glycosides phosphonates, e.g. glucosides, mannosides, and galactosides of phosphonic acid for biocompatibility.

### Examples

Objects and advantages of the particular embodiments are further illustrated by the following examples, but particular materials and amounts thereof recited in these examples, as well as other conditions and details, should not be construed to unduly limit this disclosure.

### Example 1

### Preparation of Folic Acid Nanoparticles

An aqueous solution containing approximately 5 weight percent folic acid may be prepared using purified water. Approximately, one molar NaOH solution may be added dropwise to the solution until the solution turns liquid crystalline (visually) while maintaining a pH less than 8. In this example, the pH of the solution was 6.95.

Approximately, one part by weight of the liquid crystalline solution was be combined with approximately 10 parts by weight of an approximately 10 weight percent aqueous solution of Hydroxy propyl methyl cellulose (HPMC) and the mixture was stirred using a mechanical stirrer. Approximately 1 gram (g) of this solution was added to approximately 3 milliliters (ml) of 10 weight percent aqueous solution of ZnCl₂ and the mixture was allowed to stand at room temperature without stirring for approximately 4 hours. After this time, the product mixture, comprising chromonic nanoparticles, was transferred to a poly(ethylene) centrifuge tube. The mixture was centrifuged for approximately 15 minutes and then the supernatant liquid was decanted. Purified water was added to the centrifuge tube and the mixture was again centrifuged for approximately 15 minutes. Decanting the supernatant liquid afforded the product. The product was further analyzed by dynamic light scattering using a Malvern Instruments manufactured Zen1690 particle size analyzer and was found to have a peak particle size of approximately 133 nanometers (nm).

Example 2

### Preparation of Folic Acid Nanoparticles

The procedure of Example 1 was followed; except that approximately 15 weight percent of folic acid solution was used with an aqueous solution of approximately 10% HPMC in the ratio of approximately 1:8. The product was analyzed by dynamic light scattering dynamic light scattering using a Malvern Instruments manufactured Zen1690 particle size analyzer and was found to have a mean particle size of approximately 352 nanometers.

### Example 3

### Preparation of Folic Acid Nanoparticles Including Bovine Serum Albumen (BSA)

Purified water (approximately 5 ml) was added to Fluorescein isothiocyanate conjugate albumin (fBSA, approximately 250 milligram (mg)) and then magnetically stirred for approximately 15 minutes to make a solution of approximately 50 mg fBSA/ml solution. This was mixed with a approximately 15% folic acid solution (as described in Example 1) to result in a solution that was approximately 10 weight percent of the folic acid solution and approximately 1 weight percent fBSA. The solution was stirred into a 10 weight percent aqueous solution of HPMC in a ratio of approximately 1:10 of folic acid to HPMC. After approximately 30 minutes, approximately 5 g of the mixture was added to an excess of an approximately 10 weight percent aqueous solution of ZnCl₂ and the mixture was allowed to stand. The product mixture was then transferred to a poly(ethylene) centrifuge tube. The mixture was centrifuged for approximately 20 minutes at 3200 revolutions per minute and then the supernatant liquid was decanted. Purified water was added to the centrifuge tube and the mixture was gently shaken for approximately 30 minutes and was again centrifuged for approximately 15 minutes. The supernatant liquid was decanted to provide the product.

### Example 4

### Preparation of Foliate Nanoparticles which Themselves Contain Other Nanoparticles

A portion of the chromonic mixture—fBSA was dispersed in a solution containing hydroxypropyl methylcellulose (HPMC, approximately 25% in purified water; chromonic mixture to HMPC solution ratio was 1:20 by weight) by stirring for approximately 30 minutes at room temperature.

This emulsion (approximately 0.6 g) was then added to an aqueous solution (approximately 10 ml) containing calcium chloride and zinc chloride (approximately 5% each). This solution was shaken for 30 minutes at room temperature and centrifuged at 3500 rpm for approximately 20 minutes. The resulting supernatant was then discarded. The remaining residue was washed with purified water (approximately 10 ml) and was centrifuged at 3500 rpm for approximately 20 minutes. A sample of the resulting residue fluoresced green when viewed under an optical microscope and measurements using dynamic light scattering techniques indicated that the aqueous solution contained particles in the range of 500 nm.

A portion of the resulting residue (approximately 0.6 g) was dispersed in the folic acid formulation (approximately 1 g) in purified water (approximately 1 ml). This mixture was mixed by sonicating for approximately 30 seconds and followed by stirring for approximately 20 minutes. This solution was then dispersed in HPMC (approximately 25% in purified water; foliate mixture to HMPC solution ratio 1:5 by weight) by stirring for approximately 30 minutes at room temperature. This emulsion (approximately 0.6 g) was then added to an aqueous solution (approximately 10 ml) containing calcium chloride and zinc chloride (approximately 5% each). This solution was shaken for approximately 30 minutes at room temperature and centrifuged at 3500 rpm for approximately 20 minutes. The resulting supernatant was then discarded. The remaining residue was washed with purified water (approximately 10 ml) and was centrifuged again at 3500 rpm for approximately 20 minutes. A sample of the resulting residue did not show yellow fluorescence by fBSA when viewed under an optical microscope and measurements using dynamic light scattering techniques indicated it to contain particles in the range of 1.2 to 1.5 microns. In addition, fBSA was not detected in any of the remaining wash solutions as indicated by absence of yellow fluorescence.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

What is claimed is:

1. A pteredin phenyl pentanedioic (3P) formulation comprising:
a non-covalently crosslinked aqueous solution of one or more of the 3P molecules, the 3P formulation neutralized with one of an alkali, alkali earth metal hydroxide, or alkali carbonate, the aqueous solution being crosslinked through a multivalent cation salt;
one or more of a guest molecule, surfactant, dispersant, or additive; and
a plurality of nanoparticles.

2. A method comprising:
making a 3P formulation by providing an aqueous solution comprising one of more 3P molecules neutralized with one or more of an alkali, an alkali earth metal hydroxide, or an alkali carbonate;
adding to the aqueous solution one of a surfactant, dispersant, or additive with the guest molecules; and
non-covalently crosslinking the 3P formulation by exposing the 3P formulation to an excess solution of multivalent cation salt.

3. The method of claim 2, further comprising adding to the aqueous solution one or more nanoparticles.

4. The method of claim 2, wherein the one or more 3P molecules are foliate molecules.

5. The method of claim 2, wherein the guest molecules are bioactive compounds selected from one of a drug, herbicide, pesticide, pheromone, or antimicrobial agent.

6. The method of claim 3, wherein the one or more nanoparticles comprise one or more of a metal, semiconductor, polymer, surfactant, dendrimer, lyotropic crystalline structure, liquid crystalline structure, or 3P structure.

7. The method of claim 3, wherein the one or more nanoparticles comprise a bioactive compound selected from one or more of a drug, herbicide, pesticide, pheromone, and antimicrobial agent.

8. The method of claim 7, wherein the one or more nanoparticles comprise two or more bioactive compounds that affect each other.

9. The method of claim 2, wherein a concentration of the 3P formulation has a range of approximately 0.1 to 50 weight percentage of the one or more 3P molecules.

10. The method of claim 2, wherein making the 3P formulation further comprising dispersing the 3P formulation in a water-soluble polymer phase, the water-soluble polymer phase comprising a water-soluble polymer.

11. The method of claim 3, wherein the one or more 3P nanoparticles having a dimension less than approximately 1000 nanometers.

12. The method of claim 2, further comprising contacting the crosslinked 3P formulation with a surface-modifying agent, the surface-modifying agent comprising one or more of an organic oxyacid of carbon, sulfur, phosphorus, or a combination thereof.

13. The method of claim 10, wherein a weight ratio of the water-soluble polymer phase to the 3P formulation being in a range of approximately 3:1 to 100:1.

14. The method of claim 10, wherein a concentration of the water-soluble polymer is a range of approximately 15 to 25 weight percentage of the aqueous solution.

15. The method of claim 2, wherein a multivalent cation of the multivalent cation salt is one of Ba2+, Ca²⁺, Fe.²⁺, Fe³⁺, Zn²⁺, Mg²⁺, and Al³⁺.

16. The method of claim 10, wherein the water-soluble polymer comprises one or more of a vinyl alcohol polymer, aspartic acid polymer, acrylic acid polymer, methacrylic acid polymer, acrylamide polymer, vinyl pyrrolidone polymer, poly(alkylene oxide), vinyl methyl ether polymer, sulfonated polyester, complex carbohydrate, guar gum, gum arabic, gum tragacanth, larch gum, gum karaya, locust bean gum, agar, alginate, caragheenan, pectin, cellulose, cellulose derivative, starch, modified starch, or combinations thereof.

17. A method of claim 7, wherein the bioactive compound of the one or more nanoparticles reacts with a bioactive contained in the 3P formulation.

18. The method of claim 2, further comprising:
coating a surface with the 3P formulation, the guest molecule is a salt of a noble metal;
reducing the noble metal ion to the metal; and
making arrays of nanowires or nano dots by burning off the 3P formulation.

19. The method of claim 2, further comprising exposing the 3P formulation to an aqueous solution of hydrochloric acid of pH less than approximately 4.
